(19) United States
(12) Patent Application Publication
(10) Pub. No.: US 2008/0021549 A1
(43) Pub. Date: Jan. 24, 2008

(54) Accommodating intraocular lens having an active power source

(76) Inventors: Barry T. Eagan, Spencerport, NY (US); Lloyd G. Allred, Bountiful, UT (US); Jeffrey B. McBeth, Rochester, NY (US)

Correspondence Address: Bausch & Lomb Incorporated, One Bausch & Lomb Place, Rochester, NY, 14604-2701, US

(21) Appl. No.: 11/490,895

(22) Filed: Jul. 21, 2006

(51) Int. Cl. A61F 2/16

(52) U.S. Cl. 623/6.22

(57) ABSTRACT

An accommodating intraocular lens, comprising an optical system adapted to provide variable optical power, and a drive mechanism configured to alter a focal plane of the optical system, the drive mechanism being adapted to provide at least one of a selectable speed of accommodation and disaccommodation. The drive mechanism may comprise a branch to control a speed of one of accommodation and disaccommodation.

### FIELD OF INVENTION

The present invention relates to accommodating intraocular lenses (AIOLs), and more particularly to AIOLs having an active power source.

### BACKGROUND OF THE INVENTION

There is seen in FIG. 1 a cross-sectional view of a human eye 10 having an anterior chamber 12 and a posterior chamber 14 separated by an iris 30. Within the posterior chamber 14 is a capsular bag 16 which holds the eye's natural crystalline lens 17. Light enters the eye by passing through cornea 18. The cornea and crystalline lens act together to direct and focus the light onto retina 20. The retina is connected to optic nerve 22 which transmits images received by the retina to the brain for interpretation.

In response to the sharpness of the image received by the retina, the brain operates to contract or relax ciliary muscle 26. Ciliary muscle 26 is disposed within ciliary body 28, and upon contraction of the ciliary muscle, the ciliary body is caused to move. To achieve near focus accommodation, the ciliary muscle is contracted thereby causing the ciliary body to relax tension on zonules 27 which permits the capsular bag and lens 17 to become more rounded. To achieve far focus (i.e., disaccommodation), the ciliary muscle is relaxed thereby increasing tension on zonules 27 which causes the capsular bag and lens 17 to become flatter.

In an eye where the natural crystalline lens has been damaged (e.g., clouded by cataracts), the natural lens is no longer able to properly focus and/or direct incoming light to the retina. As a result images become blurred. A well known surgical technique to remedy this situation involves removal of a damaged crystalline lens and replacement with an artificial lens known as an intraocular lens (IOL).

Conventional IOLs are typically fixed-focus lenses. Such lenses are usually selected to have a power such that the patient has a fixed focus for distance vision, and the patient requires spectacles or contact lenses to permit near vision. In recent years extensive research has been carried out to develop IOLs having variable focus capability. Such IOLs are known as accommodating IOLs (AIOLS). The term “AIOLs” refers to both single and dual optic systems.

AIOLs permit a wearer to have accommodative vision. AIOLs are typically located in the posterior chamber (e.g., in the capsular bag) and provide variable focal power in accordance with the pressure or tension exerted on the capsular bag 16 as a result of contraction and relaxation of the ciliary muscle. FIG. 1B shows an example of a two-element IOL 24 in capsular bag 16. IOL 24 comprises an anterior optic element 42 and a posterior optic element 44 that are connected to one another by haptics 46. The haptics permit optic elements 24 and 44 to translate relative to one another to achieve accommodation and disaccommodation.

AIOLs can be divided into passive AIOLs (i.e., those lenses that rely only on forces provided by the ciliary muscle to provide power to translate the one or more optics to achieve accommodation) and active AIOLs (i.e., those that rely at least in part on a battery or other active power source to provide power to translate one or more optics comprising the AIOL to achieve accommodation).

A problem with passive AIOLs that have been implanted to date is that they have provided a less than desirable amount of accommodation and have acted unpredictably when implanted in an eye. To date the reason(s) for the unpredictability and lack of accommodation in passive AIOLs has not been identified. To the best of the Applicants' knowledge no active AIOLs have been implanted to date. However, while the amount of accommodation may be more controllable than with passive AIOLs, there is reason to believe that active IOLs will suffer similar unpredictability.

### SUMMARY

Aspects of the present invention are directed to an accommodating intraocular lens, comprising an optical system adapted to provide variable optical power; and a drive mechanism configured to alter a focal plane of the optical system, the drive mechanism being adapted to provide at least one of a selectable speed of accommodation and disaccommodation. In some embodiments, the drive mechanism comprises a driver adapted to alter the focal plane of the at least one optic. The driver may be capable of changing at least one of a current, a voltage, a magnetic field, a pressure provided to the at least one optic or a position of an optic comprising the optical system. In some embodiments, a selectable speed of accommodation and/or speed of disaccommodation includes providing an operator with an ability to relatively increase or relatively decrease the speed of accommodation or disaccommodation without a knowledge of the absolute speed.

In some embodiments, the drive mechanism comprises an accommodation sensor adapted to provide an output indicative of strength of a patient's accommodative apparatus, the drive mechanism being configured to provide a speed of accommodation or disaccommodation determined at least in part on the output of the accommodation sensor.

In some embodiments, the drive mechanism comprises a branch to control a speed of one of accommodation and disaccommodation. The other of speed of accommodation and disaccommodation may be controlled by a mechanical bias of the optical system. In some embodiments, the branch comprises a first subcontroller to control the speed of accommodation or disaccommodation in response an input from an accommodation sensor adapted to provide an output indicative of strength of a patient's accommodative apparatus, and a second subcontroller. The second subcontroller may be remotely controllable.

The drive mechanism may comprise a first branch to control one of a speed of accommodation and a second branch to control speed of disaccommodation. In some embodiments, the drive mechanism comprises a remote sensor to control at least one of speed of accommodation and speed of disaccommodation.

The ciliary body (including the ciliary muscle), the zonules and the capsular bag comprise the apparatus that provide for accommodation and disaccommodation. The ciliary body (including the ciliary muscle), the zonules and the capsular bag shall be herein referred to using the term “accommodation apparatus.”

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a schematic illustration of a cross-sectional view of a human eye;

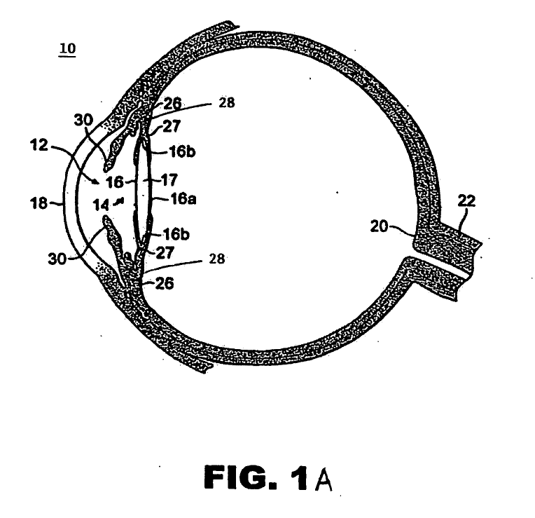

FIG. 1B is a schematic illustration of a cross-sectional view of a human eye with a two-element AIOL implanted in the capsular bag;

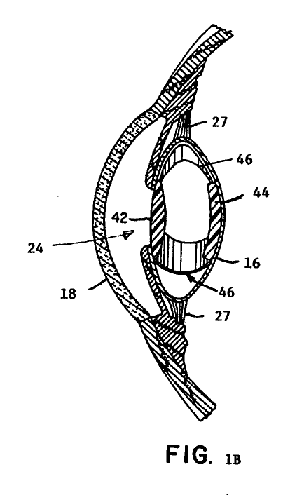

FIG. 2 is a functional block diagram of an example of an embodiment of an AIOL according to aspects of the present invention;

FIG. 3 is a schematic illustration of an example of an embodiment of an AIOL having power controllers according to aspects of invention; and

FIG. 4 is a schematic illustration of an example of an embodiment of an AIOL having an alternative configuration of a power controller branch according to aspects of invention.

### DETAILED DESCRIPTION

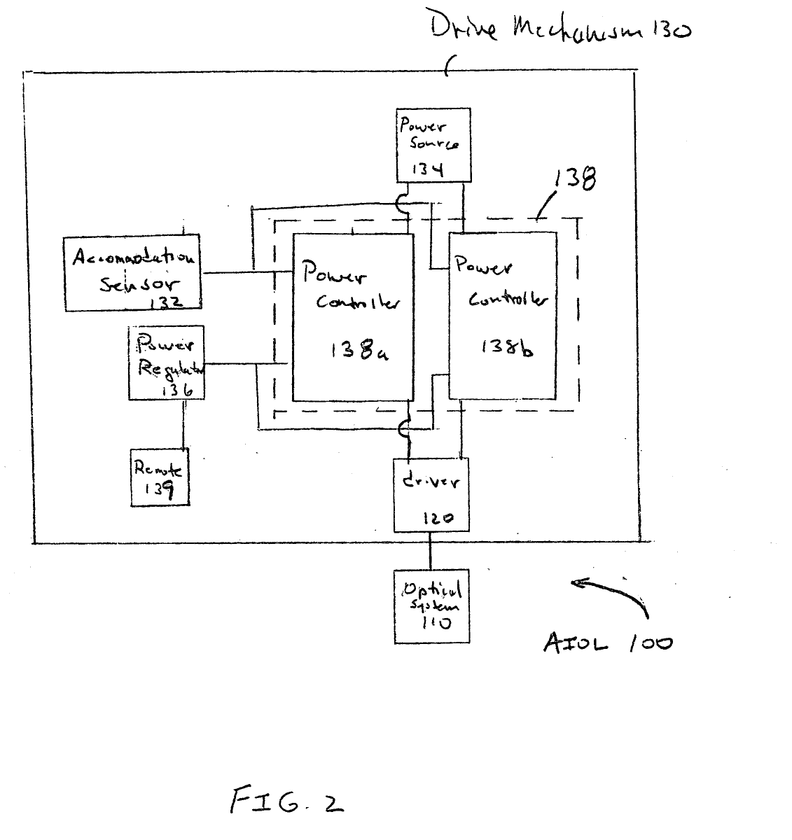

FIG. 2 is a functional block diagram of an example of an embodiment of an accommodating intraocular lens 100 according to aspects of the present invention. Accommodating intraocular lens (AIOL) 100 comprises an optical system 110 including at least one optic adapted to provide variable optical power when disposed in an eye and a drive mechanism 130 configured to alter a focal plane of the optical system, the driver mechanism being adapted to provide a selectable speed of accommodation and/or disaccommodation of AIOL 100. In the illustrated embodiment, the drive mechanism comprises a driver 120, an accommodation sensor 132, power source 134, a power controller 138, and a power regulator 136.

Drive mechanism 130 is used to increase (and/or decrease) the speed of accommodation in response to a given input provided to the accommodation sensor by a patient's eye.

It is to be appreciated that such a drive mechanism permits adaptation of an AIOL to overcome a patient's malfunctioning accommodation apparatus, as well as to overcome variations in AIOLs, such that the accommodation and/or disaccommodation occur in a suitable manner. In some embodiments, a suitable manner refers to the change in optical power being imperceivable to a patient as occurs in a normal healthy eye. However, a change in optical power provided by an IOL according to aspects of the present invention may be selected to be faster or slower than a normal healthy eye depending on the needs or desires of a given patient.

For example, a drive mechanism can overcome a patient's insufficient accommodation apparatus, which may result from aging or disease, by increasing output from driver 120 for a given output from sensor 132. Adjusting may occur at selected periods of time after implantation to treat a progressive debilitating condition (e.g., a day, a week, a month, a year or a plurality of years). Additionally, such a driver mechanism provides an ability to adjust as the accommodation apparatus heals during recovery from eye surgery.

It is also to be appreciated that such a drive mechanism permits adaptation of an AIOL to overcome variations among the population of patient's that have healthy accommodation apparatus. To overcome such variability, the AIOL can be adjusted prior to or after insertion of the AIOL into a patient to achieve a given output from driver 120 in response to an input to sensor 132 such that the accommodation and/or disaccommodation occur in a suitable manner.

Accommodation sensor 132 provides an output indicative of the strength of a patient's accommodative apparatus as the patient attempts to provide accommodation and/or disaccommodation by contracting or relaxing the ciliary muscle. Examples of suitable sensors include sensors that provide an output (e.g., a voltage or current) in response to pressure exerted on the sensor (indirectly or directly) by the ciliary muscle as it attempts to provide accommodation or disaccommodation. For example, a piezoelectric sensor may be used. In some embodiments, the piezoelectric sensor may be disposed on a haptic of the IOL, such that the IOL is squeezed by the capsular bag as the muscle attempts to provide accommodation. One example of such a device is provided in U.S. Pat. No. 4,787,903 to Grendahl, issued Nov. 29, 1988 (hereinafter Grendahl). The substance of Grendahl is hereby incorporated by reference in its entirety. Other examples of accommodation sensors include sensors capable of detecting a potential generated by the ciliary muscle as it attempts to provide accommodation. One example, of such a device is provided in U.S. Pat. No. 5,443,506 to Garabet, issued Aug. 22, 1995 (hereinafter Garabet). The substance of Garabet is hereby incorporated by reference in its entirety.

Power source 134 can be any suitable supply of power necessary to actuate driver 120. For example, the power source may include one or more implantable batteries.

Driver 120 translates at least one optic of optic system 110 or otherwise alters a characteristic of at least one element of optical system 110 to alter the focal plane of optical system 110 relative to the retina of an eye (not shown). Driver 120 controls the optical system in response to current or voltage from power source 134. The current or voltage is controlled by controller 138 as described below.

Examples of suitable drivers include drivers capable of altering a current, voltage or magnetic field applied to optical system 110 to alter the focal plane of the optical system. Examples of AIOLs including suitable driver and optical system combinations include drivers providing magnetic field outputs and liquid crystal optics as described in Grendahl and U.S. Pat. No. 6,638,304 to Azar, issued Oct. 28, 2004 (hereinafter referred to as Azar). The entirety of Azar is hereby incorporated by reference. In both Grendahl and Azar, an output of a driver is provided to a liquid crystal optic.

In other embodiments, a suitable driver 120 comprises a fluid pump driver and an optical system comprising a soft optic as described in U.S. Pat. No. 4,816,031 to Pfoff, issued Mar. 28, 1989 (hereinafter referred to as Pfoff). The pump operates to move fluid to and from a location between the optics of a two optic system. In such embodiments, the power of the optical system is altered by varying the curvature of an optic in optical system 110. The entirety of Pfoff is hereby incorporated by reference.

In yet other embodiments, a suitable driver 120 and optical system combination comprises a mechanical translation driver and one or more translatable optic elements as disclosed in Azar or U.S. Patent Appl. No. 2005/0209691 to Aharoni, et al. The entirety of Aharoni is hereby incorporated by reference (hereinafter Aharoni). In multiple-element AIOLs, the overall power of the optical system may be altered by moving one or both optics to achieve relative movement between the optics. However, the overall power of the optical system when disposed in an eye may be altered by moving only one of the optics relative the retina to alter the focal plane relative to the retina.

According to aspects of the present invention, controller 138 is adjustable such that power that is provided to driver 120 for a given output from accommodation sensor 132 is alterable. That is to say, the speed of accommodation and/or disaccommodation can be increased or decreased for a given input to the accommodation sensor from a patient's eye. For example, as discussed above, the selected speed may be altered, for example, as the ability of a patient to accommodate decreases with age (or increases after healing from surgery).

Power controller 138 is configured to control power provided to driver 120 in response to an output from accommodation sensor 132. In some embodiments, power controller 138 comprises power controller branches 138a and 138b. Power controller branch 138a controls power to achieve accommodation, and power controller branch 138b controls power to achieve disaccommodation. For example, power controller branch 138a is configured such that a positive voltage (or current) output of greater or lesser magnitude is delivered to driver 120 for a given output from sensor 132 so as to control the speed with which the power of optical system 110 is changed, to achieve faster or slower accommodation; and power controller 138b is configured such that a negative voltage (or current) output of greater magnitude is delivered to driver 120 for a given output from sensor 132 so as to control the speed with which the power of optical system 110 is changed, to achieve faster or slower disaccommodation.

Power regulator 136 provides an input to power controller 138 (i.e., one or both of power controls branches 138a and 138b) such that power that is provided to driver 120 for a given output from accommodation sensor 132 is alterable. It is to be appreciated that speed of accommodation can be thereby increased or decreased for a given output from sensor 132.

In some embodiments, a remote sensor 139 is provided to control power regulator 136. Although in some embodiments a remote sensor for controlling the speed is preferred to avoid a need for surgery to adjust the speed, in some embodiments, an adjustment device (e.g., a dial) on or proximate to the AIOL so as to be made accessible upon minimal surgery to allow adjustment of the speed of accommodation and/or disaccommodation.

In some embodiments, only a first controller branch (138a or 138b) is provided. In such embodiments, an increase in optical power of optical system 110 may be achieved by increasing current or voltage of a first polarity to driver 120, and a decrease in optical power may result from a decrease in or absence of current or voltage of the first polarity. For example, in some embodiments of optical system 110 in which the change in power is achieved by translation, driver 120 may provide an output in response to a current or voltage of a first polarity from power controller 138 so as to increase accommodation; and the lens may be configured such that an absence of such an output or a reduction in output results in the lens disaccommodating, for example, due to a mechanical bias of the lens to achieve a disaccommodated state.

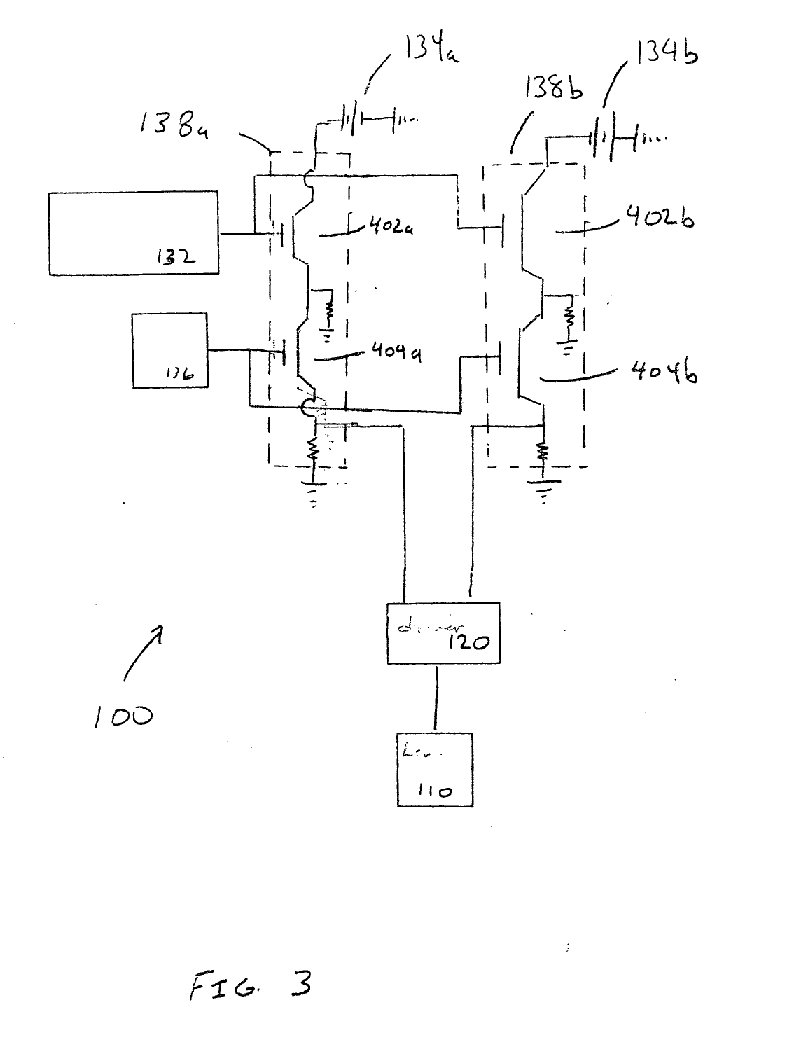

FIG. 3 is a schematic illustration of further details of an example of an embodiment of an AIOL 100 having power controller branches 138a and 138b according to aspects of invention. Power controller branch 138a is comprised of subcontrollers 402a and 404a. In the illustrated embodiment, subcontroller 402a is a transistor that controls power from power source 134 in an analog manner in response to an input from accommodation sensor 132; and subcontroller 404a is a transistor that controls power from power source 134 in response to an input from regulator 136. It will be appreciated that accommodation is thereby controlled at least in part by an output of the accommodation sensor and at least in part by an output of the regulator. Power controller 138b is comprised of subcontrollers 402b and 404b. In the illustrated embodiment, subcontroller 402b is a transistor that controls power from power source 134 in an analog manner in response to an input from accommodation sensor 132; and subcontroller 404b is a transistor that controls power from power source 134 in response to an input from regulator 136.

As discussed above, regulator 136 may be controlled remotely, thereby controlling the output from controller branches 138a and 138b and, in turn, the output to driver 120 and from driver 120, thereby controlling the speed of accommodation and/or disaccommodation.

Although subcontrollers 402a and 404a are shown as n-channel MOS transistors and subcontrollers 402b and 404b are shown as p-channel MOS transistors, the subcontrollers may be embodied as other current or voltage controllers capable of controlling the output of power source 134 reaching driver 120.

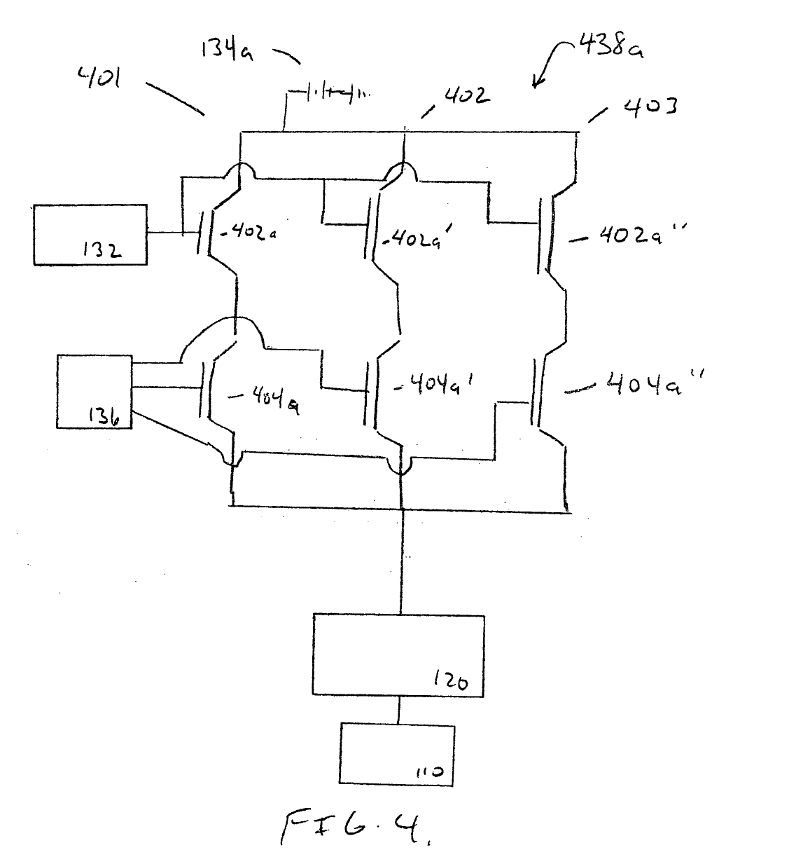

FIG. 4 is a schematic illustration of a second example of an embodiment of an AIOL having an alternative configuration of power controller branch 438a according to aspects of invention. The illustrated AIOL is the same as AIOL 100 in FIG. 3 in all aspects except in the configuration of the illustrated power controller branch. Power controller branch 138b is omitted to avoid obfuscation.

Power controller branch 438a comprises three sub-branches 401, 402 and 403. Similar to branch 138a (shown in FIG. 3) sub-branches 401, 402 and 403 each comprise a first subcontroller 402a, 402a′ and 402a″ that operates to control power from power source 134 in response to an input from accommodation sensor 132. Also, similar to branch 138a (shown in FIG. 3) sub-branches 401, 402 and 403 each comprise a second subcontroller 404a, 404a′ and 404b″ that operates to control power from power source 134 in response to an input from regulator 136. However, in power controller branch 438a each branch is controlled digitally by a digital input from regulator 136. Accordingly, each branch 401, 402 and 403 provides an output corresponding to the output in response accommodation sensor 132 to 120 if it is ON and no output if it is OFF.

Power controller branch 438a is configured such that a current from each of sub-branches 401, 402 and 403 are combined and provided to driver 120. One of ordinary skill in the art would understand that, by appropriate configuration, a voltage output from each of subbranches could be combined and provided to driver 120 to control the speed of accommodation and/or disaccommodation. Although only branch 438a is illustrated as being digitally controllable, branch 438b could be digitally controllable. For example, branch 438b could be configured with a plurality of branches in the manner illustrated in FIG. 4.

Having thus described the inventive concepts and a number of examples of embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An accommodating intraocular lens, comprising:
an optical system adapted to provide variable optical power; and
a drive mechanism configured to alter a focal plane of the optical system, the drive mechanism being adapted to provide at least one of a selectable speed of accommodation and a selectable speed of disaccommodation.

2. The accommodating intraocular lens of claim 1, wherein the drive mechanism comprises a driver adapted to alter the focal plane of the at least one optic.

3. The accommodating intraocular lens of claim 2, wherein the driver is capable of changing at least one of a current, a voltage, a magnetic field, a pressure provided to the at least one optic or a position of an optic comprising the optical system.

4. The accommodating intraocular lens of claim 1, wherein the drive mechanism comprises an accommodation sensor adapted to provide an output indicative of a strength of a patient's accommodative apparatus, the drive mechanism being configured to provide a speed of accommodation or disaccommodation determined at least in part on the output of the accommodation sensor.

5. The accommodating intraocular lens of claim 1, wherein the drive mechanism comprises a branch to control a speed of one of accommodation and disaccommodation.

6. The accommodating intraocular lens of claim 5, wherein the other of speed of accommodation and disaccommodation is controlled by a mechanical bias of the optical system.

7. The accommodating intraocular lens of claim 5, wherein the branch comprises a first subcontroller to control the speed of accommodation or disaccommodation in response an input from an accommodation sensor adapted to provide an output indicative of a strength of a patient's accommodative apparatus, and a second subcontroller.

8. The accommodating intraocular lens of claim 7, wherein the second subcontroller is remotely controllable.

9. The accommodating intraocular lens of claim 1, wherein the drive mechanism comprises a first branch to control one of a speed of accommodation and a second branch to control speed of disaccommodation.

10. The accommodating intraocular lens of claim 1, wherein the drive mechanism comprises a remote sensor to control at least one of speed of accommodation and speed of disaccommodation.

11. The accommodating intraocular lens of claim 1, wherein the drive mechanism is adapted to provide both a selectable speed of accommodation and a selectable speed of disaccommodation
